(19) United States
(12) Patent Application Publication
(10) Pub. No.: US 2025/0328836 A1
(43) Pub. Date: Oct. 23, 2025

(54) MANAGEMENT SYSTEM, MANAGEMENT METHOD, AND STORAGE MEDIUM

(71) Applicant: TOYOTA JIDOSHA KABUSHIKI KAISHA, Toyota-shi (JP)

(72) Inventors: Satoshi TOYOSHIMA, Toyota-shi (JP); Shiro Oda, Anjo-shi (JP); Takeshi Matsui, Nisshin-shi (JP)

(73) Assignee: TOYOTA JIDOSHA KABUSHIKI KAISHA, Toyota-shi (JP)

(21) Appl. No.: 18/990,212

(22) Filed: Dec. 20, 2024

(30) Foreign Application Priority Data
Apr. 22, 2024 (JP) 2024-068919

(51) Int. Cl. G06Q 10/0631

(52) U.S. Cl. CPC G06Q 10/06312; G06Q 10/06311

(57) ABSTRACT

The management system according to the present embodiment is a management system including a server that manages a schedule including a conveying task using a plurality of robots in a facility, wherein the server acquires arrival schedule information indicating an arrival schedule time and an arrival schedule location of a conveying target to the facility, the server specifies a type of robot that can transfer an arrival scheduled transfer target based on the arrival schedule information, and the server updates a schedule of another robot so that the specified robot can perform reception at the arrival schedule location in accordance with the arrival schedule time of the conveying target.

### CROSS-REFERENCE TO RELATED APPLICATION

This application claims priority to Japanese Patent Application No. 2024-068919 filed on Apr. 22, 2024, incorporated herein by reference in its entirety.

### BACKGROUND

### 1. Field

The present disclosure relates to a management system, a management method, and a storage medium.

### 2. Description of Related Art

Japanese Unexamined Patent Application Publication No. 2023-81320 (JP 2023-81320 A) discloses a disclosure system that manages an autonomous vehicle in a hospital. In a hospital, an autonomous vehicle carries medical supplies, drugs, linen, specimens, meals and the like.

### SUMMARY

Incidentally, when a robot such as an autonomous vehicle carries a conveyed object, there is a desire to perform a conveying service (also called a conveying task) more efficiently.

A management system relating to the present aspect is

a management system including a server that manages a schedule including a conveying task using a plurality of autonomous mobile robots in a facility,
the server acquires arrival schedule information that indicates an arrival schedule time and an arrival schedule location of a conveying target to the facility,
the server specifies an autonomous mobile robot of a kind that is able to perform conveyance of the conveying target to be arrived based on the arrival schedule information, and
the server updates a schedule of the specified autonomous mobile robot and a schedule of another autonomous mobile robot such that the specified autonomous mobile robot is able to execute reception at the arrival schedule location in accordance with the arrival schedule time of the conveying target.

A management method relating to the present aspect is

a management method that manages a schedule including a conveying task using a plurality of autonomous mobile robots in a facility by using a computer, the management method including
acquiring arrival schedule information that indicates an arrival schedule time and an arrival schedule location of a conveying target to the facility,
specifying an autonomous mobile robot of a kind that is able to perform conveyance of the conveying target to be arrived based on the arrival schedule information, and
updating a schedule of the specified autonomous mobile robot and a schedule of another autonomous mobile robot such that the specified autonomous mobile robot is able to execute reception at the arrival schedule location in accordance with the arrival schedule time of the conveying target.

A storage medium relating to the present aspect is

a storage medium storing a program for causing a computer to execute a management method that manages a schedule including a conveying task using a plurality of autonomous mobile robots in a facility,
the management method includes
acquiring arrival schedule information that indicates an arrival schedule time and an arrival schedule location of a conveying target to the facility,
specifying an autonomous mobile robot of a kind that is able to perform conveyance of the conveying target to be arrived based on the arrival schedule information, and
updating a schedule of the specified autonomous mobile robot and a schedule of another autonomous mobile robot such that the specified autonomous mobile robot is able to execute reception at the arrival schedule location in accordance with the arrival schedule time of the conveying target.

According to the present disclosure, a management system, a management method, and a storage medium can be provided that can convey a conveying target more efficiently.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a schematic diagram illustrating an overall configuration of a management system according to the present embodiment;

FIG. 2 is a block diagram illustrating a control system of the management apparatus;

FIG. 3 is a time table showing schedules before and after coordination; and

FIG. 4 is a flowchart illustrating a management method.

### DETAILED DESCRIPTION OF EMBODIMENTS

Hereinafter, the present disclosure will be described through embodiments of the disclosure. However, the disclosure according to the claims is not limited to the following embodiments. Moreover, all of the configurations described in the embodiments are not necessarily indispensable as means for solving the issue.

### Overall Configuration

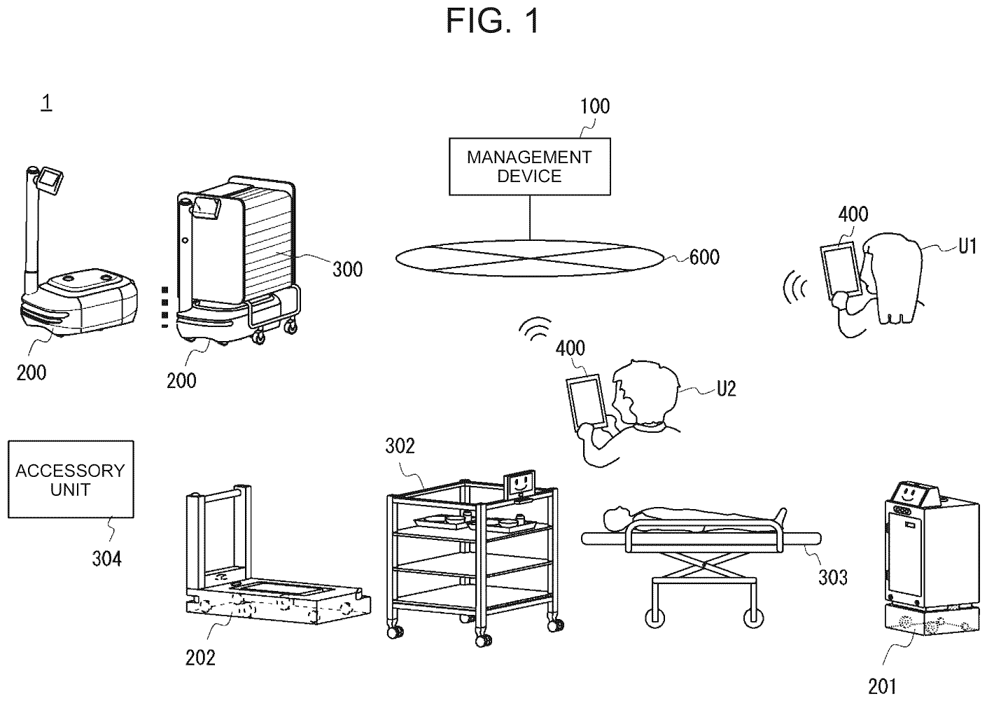

The management system according to the present embodiment is a system for managing an autonomous mobile robot capable of executing a transport service (also referred to as a conveying task). FIG. 1 is a schematic diagram illustrating a configuration of a management system 1. The management system 1 includes a management device 100, robots 200 to 202, accessory units 300, 302, 303, and 304, a network 600, and a user terminal 400. The management system 1 is a system for managing a plurality of robots 200 to 202 and accessory units 300, 302, 303, and 304 thereof. The management device 100 manages schedules of the plurality of robots 200 to 202 and the plurality of accessory units 300, 302, 303, and 304.

The robots 200 to 202 are autonomous mobile robots, and execute transport services. The robot 200 autonomously moves medical welfare facility such as hospitals, rehabilitation centers, nursing facility, and residential facility for the elderly. The robots 200 to 202 are used for transportation of drugs, medical devices, meals, tableware, medical records, fixtures, samples, linens, persons, and the like. The conveying target may be a person, such as a patient. Moreover, the system according to the present embodiment can also be used in commercial facility such as shopping malls. The robots 200 to 202 include wheels, a chassis, a motor, a sensor, a battery, a controller, and the like. The robot 200, the robot 201, and the robot 202 are different types of robots. The robots 200 to 202 are assigned unique identifying numbers (ID).

The robot 200 executes the conveyance service using the accessory unit 300. The accessory unit 300 is a conveyance unit used in combination with the robot 200 for conveyance of a conveyed object. The accessory unit 300 is a wagon or a cart with wheels, in which a conveyed object can be mounted. The bogie portion of the robot 200 serves as a stage on which a wagon or the like is mounted.

The robot 200 has a lifting function for lifting the accessory unit 300. When the bogie portion of the robot 200 enters the lower side of the accessory unit 300, an elevating stage or the like lifts the accessory unit 300. Thus, the robot 200 mounts the accessory unit 300. The robot 200 can convey the conveyed object accommodated in the accessory unit 300 to the destination. The robot 200 can execute the conveyance service by mounting the accessory unit 300. The accessory unit 300 for transportation is used for serving a table in a restaurant, a medical welfare facility, or the like, or for a lower table. The transport unit may be used for transporting parts in a factory, transporting samples in a hospital, transporting linen in a hotel, and the like.

The robot 201 is a transfer robot that independently executes a transfer service. For example, the robot 201 includes a storage unit or the like that stores a conveyed object, and conveys the conveyed object without using the accessory unit 300. The robot 201 can lock the accommodation portion in order to safely convey the conveyed object.

The robot 202 executes the conveyance service using the accessory unit 302. The accessory unit 302 is a conveyance unit used in combination with the robot 202 for conveyance of a conveyed object. Specifically, the accessory unit 302 is a wagon or cart with wheels, and has a plurality of shelves. For example, the accessory unit 302 is used for serving a table or a lower table. Therefore, the tableware, which is the conveyed article, is placed on each stage of the accessory unit 302. Of course, the accessory unit 302 may be loaded with a conveyed article other than tableware.

The robot 202 is a bogie-type moving body. Similar to the robot 200, the robot 202 has an elevating function for lifting the accessory unit 302. When the bogie portion of the robot 202 enters the lower side of the accessory unit 302, an elevating stage or the like lifts the accessory unit 302. Thus, the robot 202 mounts the accessory unit 302. The robot 202 can transport the tableware placed on the accessory unit 302 to a destination. The robot 202 is equipped with the accessory unit 302 so that a transportation service such as a table setting or a lower table can be executed.

In addition, the robot 202 can perform a transport service using the accessory unit 303. Here, the accessory unit 303 includes a stretcher, a support, and the like for transporting a person such as a patient. Then, a person rides on the accessory unit 303. The robot 202 enters the lower side of the accessory unit 303, and the lifting stage lifts the accessory unit 303. A robot 202 mounts an accessory unit 303. As a result, the robot 202 can transport the person to the destination. The robot 202 uses the accessory unit 302 or the accessory unit 303 depending on the conveyed object to be conveyed.

Note that the management system 1 may include a robot that conveys a person independently without using the accessory unit 303. In this case, the robot may be a support-type transport robot or a wheelchair-type transport robot. The management system 1 may include a stretcher conveying robot, a wheelchair robot, and a walking assistance robot and the like. The conveyed object is not limited to an object, and includes a person. Further, a conveyed object which is an object or a person is also referred to as a conveying target.

Further, at least one of the robots 200 to 202 may execute other services other than the transportation service. Other services include cleaning services, security services, and guidance services. Here, the robot 200 executes a plurality of services such as cleaning, security, and guidance using the accessory unit 304. The robot 200 executes various services by using the accessory unit 300 in combination with the robot 200. The robot 200 is provided with different accessory units 304 depending on the service. By replacing the accessory unit 304, the robot 200 becomes a multi-service robot that executes 25 a plurality of services.

The accessory unit 304 includes a cleaning unit for cleaning, a security unit for security, and a guide unit for guidance. The robot 200 mounts any one of the accessory units. That is, the robot 200 can attach and detach the accessory unit 300. The robot 200 is equipped with an accessory unit 300 corresponding to a service to be executed. The robot 200 switches between the accessory unit 300 and the accessory unit 304 according to the service to be executed.

The accessory unit 304 for cleaning is a unit for cleaning a facility. The accessory unit 304 for cleaning includes a cleaner for sucking dust and the like. Alternatively, the cleaning accessory unit 304 includes a brush, a pad for wiping the floor, a mop, and the like. When the robot 200 moves with the accessory unit 304 for cleaning attached thereto, the floor surface is cleaned. That is, in the region where the robot 200 has moved, the floor surface is cleaned. The robot 200 can execute the cleaning service by mounting the accessory unit 304 for cleaning. The accessory unit 304 for cleaning may also be a remotely controllable robotic cleaner. In this case, the robot 200 performs the cleaning service by remotely controlling the accessory unit 304 for cleaning by wireless communication.

The accessory unit 304 for security is a unit for security of a facility. The accessory unit 304 for security includes, for example, various sensors for detecting an intruder or an abnormality. For example, the security accessory unit 304 includes a camera, an infrared camera, a ranging sensor, an optical sensor, a thermal sensor, and a smoke sensor as sensors. A lighting device for illuminating an intruder or an abnormal portion may be provided. The accessory unit 304 may have an alarm function or the like for notifying when an abnormality is detected. The robot 200 can execute the security service by wearing the accessory unit 304 for security.

The guide accessory unit 304 is a unit for guiding a facility. For example, the accessory unit 304 for guidance guides a visitor of the facility to a route to the destination. The guide accessory unit 304 has an input device for the visitor to input the destination. The input device includes a touch panel and buttons. The input device also includes a microphone for voice input. The robot 200 moves to a destination to guide the visitor. The guide accessory unit 304 may include a display device for displaying a route and a speaker for outputting a voice of the route.

As described above, when the accessory unit 304 is used in combination with the robot 200, a plurality of different services can be executed by the robot 200. That is, the robot 200 executes a service corresponding to the accessory unit 304 mounted thereon. Of course, the types of robots that can be introduced are not limited to two types. For example, the type of the robot may be one type or three or more types. In addition, a robot that executes a service other than the conveyance service may be introduced. A dedicated robot that performs various services such as a cleaning service, a security service, and a guidance service may be introduced.

As described above, when the accessory unit 304 is used in combination with the robot 200, a plurality of different services can be executed by the robot 200. That is, the robot 200 executes a service corresponding to the accessory unit 304 mounted thereon. accessory units 300, 302, 303, and 304 are assigned unique ID. Of course, at least some of the services may be executed by the robot 200 alone. Further, the robots 201 and 202 may execute services other than the conveyance service.

The management system 1 may be provided with a plurality of accessory units 304 for one service. Two or more accessory units 300 may be provided in the management system 1. In this case, the two robots 200 can simultaneously execute the transport service using the accessory unit 300. Of course, two or more accessory units 300, accessory units 302, or accessory units 303 may be provided. Further, a plurality of types of accessory units may be prepared for the service performed by one robot. For example, the shape and size of the accessory unit 300 may be different depending on the contents of the conveyed material.

The user U1 or the user U2 can use the user terminal 400 to make a service request such as a conveyance request for a conveyed object. For example, the user terminal 400 is a tablet computer, smart phone, or the like. The user terminal 400 only needs to be an information processing device capable of wireless or wired communication.

In the present embodiment, the robots 200 to 202 and the user terminal 400 are connected to the management device 100 via the network 600. The network 600 is a wired or wireless local area network (LAN) or wide area network (WAN). Further, the management device 100 is connected to the network 600 by wire or wirelessly. For example, the communication between the devices may be based on a general-purpose communication standard such as Wi-Fi (registered trademark).

Various types of signals transmitted from the user terminal 400 of the user U1, U2 are once transmitted to the management device 100 via the network 600 and transferred from the management device 100 to the target robots 200 to 202. Similarly, various signals transmitted from the robots 200 to 202 are once transmitted to the management device 100 via the network 600, and transferred from the management device 100 to the target user terminal 400. The management device 100 is a server connected to each device, and collects data from each device. The management device 100 is not limited to a physically single apparatus, and may include a plurality of apparatuses that perform distributed processing. In addition, the management device 100 may be distributed and arranged in an edge device such as the robots 200 to 202. For example, some or all of the management system 1 may be mounted on the robots 200 to 202.

The user terminal 400 and the robots 200 to 202 may transmit and receive signals without passing through the management device 100. For example, the user terminal 400 and the robots 200 to 202 may transmit and receive signals directly by wireless communication. The management device 100 may collect data from a monitoring camera or a communication device (not shown).

It is assumed that a plurality of types of robots 200 to 202 are used in a facility. The management device 100 assigns services to the respective robots 200 to 202. Each of the robots 200 to 202 is equipped with an accessory unit 300 corresponding to the assigned service and executes the service. The services executed by the robots 200 to 202 may be entered by a user U1 or a user U2, or may be pre-scheduled. For example, a user U1 or the like operates the user terminal 400 to make a service request. A user U1 or the like can enter the type of service to be executed. A user U1 or the like may enter an area, a time-zone, or the like in which the service is executed. The management device 100 creates a schedule for the robots 200 to 202 to efficiently execute the service.

The user U1 or the user U2 may operate the user terminal 400 to request a transport service. Here, the user U1 or the user U2 enters information about the conveyed object. Further, the user U1 or the user U2 may enter the arrival schedule information indicating the arrival schedule of the conveyed object. The management device 100 allocates a robot that executes the transport service based on the arrival schedule information. Then, the management device 100 transmits a control signal for the robot to execute the service.

In such an overall configuration, each element of the management system 1 can be distributed among the robots 200 to 202, the user terminals 400, and the management device 100 to construct the management system 1 as a whole. Further, it is possible to collect substantial elements for achieving the transportation of the transported object in a single device to construct the system.

### Control System

The management device 100 includes a server computer and the like, and performs arithmetic operations for controlling and managing the robots 200 to 202. The management device 100 can be implemented as, for example, a program-executable device such as a central processing unit (CPU: Central Processing Unit) of a computer. The functions described later can also be realized by a program. The management device 100 manages the conveyed object ID of the conveyed object, the unit ID of the accessory units 300, 302, 303, and 304, and the robot ID of the robots 200 to 202.

For example, the management device 100 manages schedules of the plurality of robots 200 to 202 so that the robots 200 to 202 can efficiently execute services. For example, when receiving a service request from the user terminal 400 or the like, the management device 100 selects one robot 200 from the plurality of robots 200 to 202 and instructs the robot 200 to execute the service. Alternatively, the management device 100 instructs the robot 200 to use the accessory unit 300.

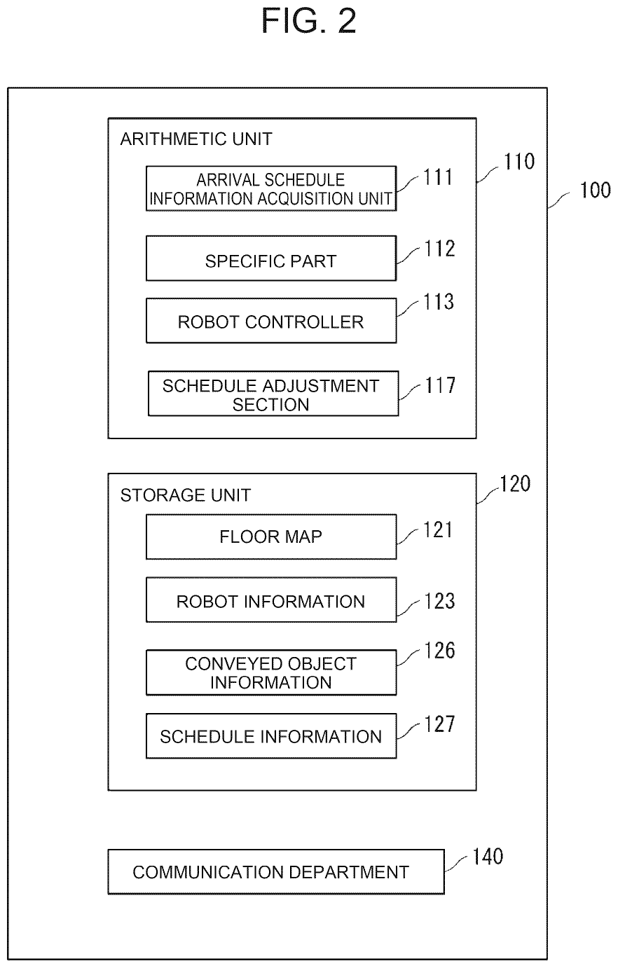

FIG. 2 is a block diagram illustrating a control system of the management device 100 according to the present embodiment. As illustrated in FIG. 2, the management device 100 includes an arithmetic processing unit 110, a storage unit 120, and a communication unit 140. The arithmetic processing unit 110 includes an arrival schedule information acquisition unit 111, an identification unit 112, a robot control unit 113, and a schedule adjustment unit 117.

The arithmetic processing unit 110 may perform arithmetic processing other than these. For example, the arithmetic processing unit 110 may perform route planning or the like according to the schedule of each robot. In this case, the management device 100 transmits a movement path to each robot. Of course, the robot may perform route planning. In this case, when the management device 100 transmits a route, a destination, or the like to the robot, the robot performs route planning.

The storage unit 120 stores a floor map 121, robot information 123, conveyed object information 126, and schedule information 127. FIG. 2 shows a part of the information stored in the storage unit 120, and information other than the information shown in FIG. 2 may be stored in the storage unit 120. For example, the storage unit 120 may store programs, control parameters, and the like.

The floor map 121 is map information of a facility. The floor map 121 may include information about an arrival schedule location and the like, which will be described later. For example, the floor map 121 may be created in advance. In addition, the floor map 121 may be not map information of the entire facility but map information partially including an area in which a service is to be executed. Each robot autonomously travels to the destination by referring to the floor map.

The robot information 123 includes information about the robots 200 to 202 operating in the facility. The robot information 123 includes information about the model numbers of the robots 200 to 202, an executable service, a transportable conveyance type, an accessory unit, and the like. The storage unit 120 stores the robot information 123 as a data base in which various kinds of information and the like are stored for each robot ID. The robot information 123 may include a current position of the robot, a moving route, information indicating whether the service is being executed or suspended, and information regarding the service being executed. The robot information 123 may include information about an accessory unit in use or a conveyed object being conveyed.

The conveyed object information 126 includes information on the conveyed object. For example, the conveyed object information 126 includes information such as an identification number (ID) of the conveyed object, a content (type) of the conveyed object, a conveyance source, a conveyance destination, a reception time, or an arrival time. The conveyed object information 126 is information indicating whether the conveyed object is a medicine, a medical device, a meal, a tableware, a medical record, a fixture, a specimen, a linen, or a person. The conveyed object information 126 may include information such as a size or a weight of the conveyed object. The conveyed object information 126 may include information indicating a status such as during conveyance, before conveyance (before mounting), and after conveyance. The storage unit 120 stores the conveyed object information 126 as a data base in which these pieces of information are associated with each conveyed object ID. When a new conveyance request is received from the user terminal 400, conveyance information is added. Further, after the completion of the conveyance, the information on the conveyed object may be deleted from the list.

The schedule information 127 includes information indicating schedules of the plurality of robots 200 to 202. For example, the schedule information 127 includes information about a service executed by each robot. The schedule information 127 includes information on the type of service, the scheduled start time, and the scheduled end time. The schedule information 127 may include information about an area or a point where the service is to be performed. For example, the schedule information 127 may include information regarding a receiving location of a conveyed object or a location of a conveyance destination. The storage unit 120 stores the above-described information for each robot as the schedule information 127 as a database. Schedule information 127 may include information about an accessory unit used by the robot.

The arrival schedule information acquisition unit 111 acquires the arrival schedule information. The arrival schedule information is information indicating an arrival schedule of a conveyed object (also referred to as a conveying target) to the facility. The arrival schedule information includes information on the arrival schedule time and the arrival schedule location of the conveyed object. Further, the arrival schedule information includes information on the type of the conveyed object (conveyed object type). Further, the arrival schedule information may include information such as a transport destination. For example, the user U1 may operate the user terminal 400 to enter arrival schedule times, arrival schedule locations, arrival schedule information of expected delivery types, and the like. The arrival schedule information acquisition unit 111 acquires the arrival schedule information before the arrival schedule time to the facility.

For example, the scheduled collection time and the collection location of the specimen collected from the patient are the arrival schedule time and the arrival schedule location. Alternatively, when the patient is transported to the hospital by an ambulance or the like, the time when the patient arrives at the hospital and the acceptance place become the arrival schedule time and the arrival schedule location of the transported object. In addition, the user U1 may enter information indicating whether the conveyed object is an urgent conveyed object or a priority order of conveyance. Then, the following schedule adjustment processing may be executed only for the conveyed object that needs to be urgently conveyed. Of course, the arrival schedule information may be an automatic input by a computer.

The identification unit 112 specifies a type of robot that is capable of transporting an arrival-scheduled conveying target based on the arrival schedule information. For example, the identification unit 112 specifies a robot capable of transferring the conveyed object based on the conveyed object type. As described above, the conveyed objects that can be conveyed are different for each robot. The identification unit 112 refers to the robot information 123 and specifies a robot capable of transporting the conveyed object. The identification unit 112 may specify the robot to be transported by referring to the floor map 121, the schedule information 127, or the robot information 123. A robot that is specified by the identification unit 112 so as to convey the conveyed object indicated in the arrival schedule information is referred to as a specific robot.

When there are two or more robots capable of transporting the conveyed object, the identification unit 112 specifies the specified robot so that the transportation service can be executed more efficiently. For example, the identification unit 112 refers to the schedule information 127 and the robot information 123 to specify a robot in the vicinity of the arrival schedule location at the arrival schedule time. Further, the identification unit 112 specifies a free-state robot in which another service is not executed at the arrival schedule time. Alternatively, the identification unit 112 refers to the robot information 123 and specifies a robot having an empty space in a storage location such as the accessory unit 300

The robot control unit 113 controls the robots 200 to 202 to execute the scheduled service. For example, a control signal is generated so as to be attached to an accessory unit 300 or the like for executing a service. Alternatively, the robot control unit 113 generates a control signal for moving the robot 200 to the destination so as to execute the service.

The schedule adjustment unit 117 adjusts the schedules of the plurality of robots based on the arrival schedule information. For example, with reference to the schedule information 127, the specific robot is moved to the arrival schedule location at the arrival schedule time indicated in the arrival schedule information. In this way, the specific robot can move to the arrival schedule location indicated by the arrival schedule information by the arrival schedule time. Since the specific robot is waiting at the arrival schedule location, it is possible to shorten the period in which the conveying target stands by at the facility.

For example, a carrier that transports a package or the like of a conveying target can increase the efficiency of the transportation schedule. In addition, it is possible to shorten the time required for a conveying target person to receive an appropriate treatment at a facility. At the arrival schedule location, the robot can quickly load and transport the transported object. Therefore, the conveyed object can be efficiently conveyed. Further, even in a time zone where the number of employees is small, such as at midnight or on holidays, the conveyed object can be appropriately and efficiently conveyed.

Further, in a case where another service is allocated to the specific robot at the arrival schedule time or before or after the arrival schedule time, the schedule adjustment unit 117 changes the schedule so that the other robot executes the service. That is, the schedule adjustment unit 117 assigns a service that the specific robot is scheduled to execute to another robot. The schedule adjustment unit 117 updates the schedules of the specific robot and the robots other than the specific robot so that the specific robot can perform reception at the arrival schedule location in accordance with the arrival schedule time of the conveyed object.

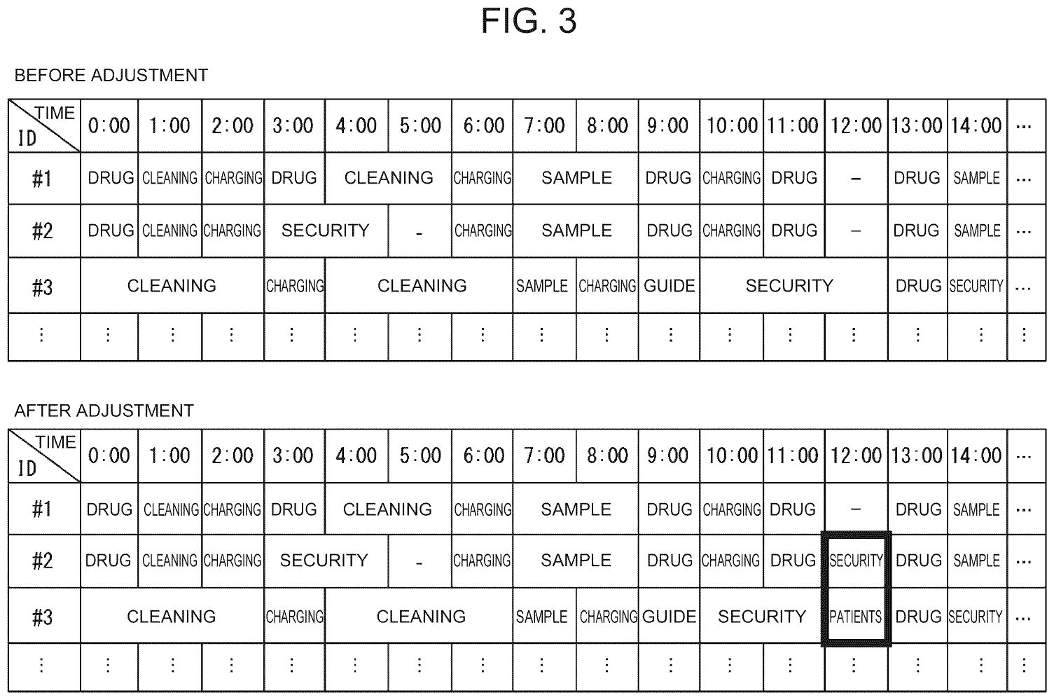

The schedule adjustment processing will be described with reference to FIG. 3. FIG. 3 is a time table showing schedules before and after adjustment. FIG. 3 shows a schedule of three robots. Hereinafter, ID of the three robots will be described as #1-#3.

The robot of #1 can execute cleaning service and conveyance service. The #1 robot is capable of carrying specimens and chemicals in the transport service. The robot with #1 cannot carry linen, people, etc. The robot with #1 cannot perform security service. The #2 robot can perform cleaning services, security services, and conveyance services. The #2 robot is capable of transporting specimens and chemicals in the transport service. #2 robot cannot transport linen, people, etc. The robot of #3 can perform cleaning services, guidance services, security services and conveyance services. The #3 robot is capable of transporting specimens, chemicals, and patients (people) in the transportation service.

In FIG. 3, charging indicates that each robot is charging a battery. During charging, the robot is stopping at the charging station, so the service is not running. It is assumed that the arrival schedule information acquisition unit 111 acquires the arrival schedule information indicating that the patient (person) is scheduled to arrive at 12:00. Here, since only the robot #3 can transport a person, the schedule adjustment unit 117 changes the schedule so as to assign the conveying task of the conveyed object indicated by the arrival schedule information to the robot #3. Further, the security service assigned to the robot #3 before the schedule adjustment is assigned to the robot #2.

In this way, even when there is an emergency conveyance request, the entire system can be efficiently conveyed. In addition, the schedule is updated so that other services such as security services and transportation of other conveyed objects are assigned to robots other than the specific robot. Therefore, the service can be efficiently executed as a whole system.

The description of FIG. 2 is returned. The communication unit 140 includes a communication interface for communicating with the user terminal 400 or the robots 200 to 202. The communication unit 140 transmits a control signal and schedule information 127 for controlling the robot to the robot. The communication unit 140 may transmit information such as a destination and a moving route to the robot. The communication unit 140 receives a service request from the user terminal 400.

The management system 1 may be introduced into a facility such as a medical welfare facility. When the conveying target indicated by the arrival schedule information is a person, the type of regular moving robot that can convey the conveying target scheduled to arrive may be any one or a plurality of a stretcher conveying robot, a stretcher conveying assistance robot, a wheelchair robot, and a walking assistance robot. This makes it possible to quickly convey a person who needs to be urgently conveyed. For example, an emergency patient carried by an ambulance or a patient whose condition has changed suddenly can be rapidly transported.

The at least one autonomous mobile robot may comprise a main subject robot and an accessory unit. The main subject robot operates autonomously as a subject. The accessory unit is used in combination with the main subject robot to enable the main subject robot to carry a predetermined type of conveying target. As a result, the robot can convey various conveyed objects, and thus convenience can be improved.

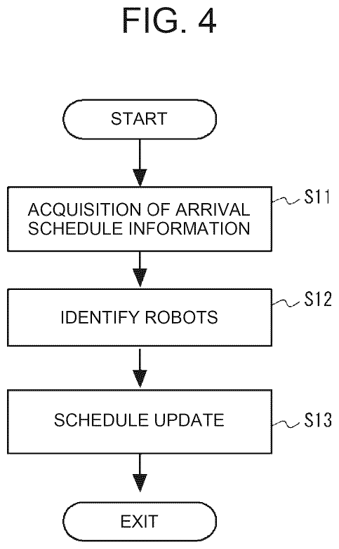

FIG. 4 is a flowchart illustrating a management method. The arrival schedule information acquisition unit 111 acquires arrival schedule information indicating the arrival schedule times and arrival schedule locations of the conveyed objects (S11). The identification unit 112 specifies a robot that can convey the conveyed object (S12). The schedule adjustment unit 117 S13 the schedule of the specified robot and other robots. By doing so, it is possible to efficiently execute the transport service.

The management device 100 and the robots 200 to 202 may use a machine learning model such as deep learning in route planning and drive control. Further, in detection of a surrounding object or the like, a machine learning model such as deep learning such as RNN (Recurrent Neural Network) or CNN (Convolutional neural network) may be used.

In addition, some or all of the processes in the robots 200 to 202, the management device 100, and the like described above can be realized as a computer program. The program as described above is stored using various types of non-transitory computer-readable media, and can be supplied to a computer. The non-transitory computer-readable media include various types of tangible recording media (storage media). Examples of the non-transitory computer-readable media include magnetic recording media (e.g. flexible disks, magnetic tapes, hard disk drives), magneto-optical recording media (e.g. magneto-optical disks), compact disc read-only memory (CD-ROM), compact disc recordable (CD-R), compact disc rewritable (CD-R/W), and semiconductor memory (e.g. mask ROM, programmable ROM (PROM), erasable PROM (EPROM), flash ROM, random access memory (RAM)). Further, the program may also be supplied to the computer by various types of transitory computer-readable media. Examples of the transitory computer-readable media include electrical signals, optical signals, and electromagnetic waves. The transitory computer-readable media can supply the program to the computer via a wired communication path such as an electric wire and an optical fiber, or a wireless communication path.

The present disclosure is not limited to the above embodiment, and can be appropriately modified without departing from the spirit.

What is claimed is:

1. A management system comprising a server that manages a schedule including a conveying task using a plurality of autonomous mobile robots in a facility, wherein
the server acquires arrival schedule information that indicates an arrival schedule time and an arrival schedule location of a conveying target to the facility,
the server specifies an autonomous mobile robot of a kind that is able to perform conveyance of the conveying target to be arrived based on the arrival schedule information, and
the server updates a schedule of the specified autonomous mobile robot and a schedule of another autonomous mobile robot such that the specified autonomous mobile robot is able to execute reception at the arrival schedule location in accordance with the arrival schedule time of the conveying target.

2. The management system according to claim 1, wherein when the facility is a medical welfare facility and the conveying target indicated by the arrival schedule information is a person, the autonomous mobile robot of the kind that is able to perform conveyance of the conveying target to be arrived is one or more of a stretcher conveying robot, a stretcher conveying assistance robot, a wheelchair robot, and a walking assistance robot.

3. The management system according to claim 1, wherein at least one autonomous mobile robot of the autonomous mobile robots includes
a main subject robot that operates autonomously as a main subject, and
an accessory unit used in combination with the main subject robot such that the main subject robot executes conveyance of the conveying target of a predetermined kind.

4. A management method that manages a schedule including a conveying task using a plurality of autonomous mobile robots in a facility by using a computer, the management method comprising:
acquiring arrival schedule information that indicates an arrival schedule time and an arrival schedule location of a conveying target to the facility;
specifying an autonomous mobile robot of a kind that is able to perform conveyance of the conveying target to be arrived based on the arrival schedule information; and
updating a schedule of the specified autonomous mobile robot and a schedule of another autonomous mobile robot such that the specified autonomous mobile robot is able to execute reception at the arrival schedule location in accordance with the arrival schedule time of the conveying target.

5. A non-transitory storage medium storing a program for causing a computer to execute a management method that manages a schedule including a conveying task using a plurality of autonomous mobile robots in a facility, wherein the management method includes
acquiring arrival schedule information that indicates an arrival schedule time and an arrival schedule location of a conveying target to the facility,
specifying an autonomous mobile robot of a kind that is able to perform conveyance of the conveying target to be arrived based on the arrival schedule information, and
updating a schedule of the specified autonomous mobile robot and a schedule of another autonomous mobile robot such that the specified autonomous mobile robot is able to execute reception at the arrival schedule location in accordance with the arrival schedule time of the conveying target.
